(19) United States
(12) Patent Application Publication
(10) Pub. No.: US 2026/0165873 A1
(43) Pub. Date: Jun. 18, 2026

(54) FLUID COLLECTION DEVICES, SYSTEMS, AND METHODS

(71) Applicant: PUREWICK CORPORATION, Covington, GA (US)

(72) Inventor: Kathleen Davis, Atlanta, GA (US)

(21) Appl. No.: 19/416,087

(22) Filed: Dec. 11, 2025

Related U.S. Application Data
(63) Continuation of application No. 18/044,413, now Pat. No. 12,521,272.
(60) Provisional application No. 63/076,032, filed on Sep. 9, 2020.

(51) Int. Cl. A61F 5/455; A61F 5/44

(52) U.S. Cl. CPC A61F 5/455; A61F 5/4401; A61F 5/4404

(57) ABSTRACT

Examples relate to systems, devices, and methods for collecting fluid from wearer of the fluid collection device. The fluid collection devices include porous material having one or more raised portions to fit within the female anatomy near the urethra. A fluid collection system includes a fluid collection device operably coupled to a fluid storage container and vacuum source via one or more sections of conduit.

### CROSS REFERENCE TO RELATED APPLICATIONS

This application is a continuation of U.S. application No. Ser. No. 18/044,413 filed on Mar. 8, 2023, which is a U.S. Nationalization of PCT International Application No. PCT/US2021/049404 filed on Sep. 8, 2021, which claims priority to U.S. Provisional Application No. 63/076,032 filed on Sep. 9, 2020, the disclosure of each of which is incorporated herein in its entirety by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may uncomfortable, painful, and may cause urinary tract infections.

Thus, users and manufacturers of fluid collection devices continue to seek new and improved devices, systems, and methods to collect urine.

### SUMMARY

Embodiments disclosed herein are related to devices, systems, and methods of using fluid collection devices. In an embodiment, a fluid collection device is disclosed. The fluid collection device includes a fluid impermeable barrier having an outer surface and an inner surface, the inner surface at least partially defining a chamber, the fluid impermeable barrier also defining an opening extending therethrough, the opening configured to be positioned adjacent to a female urethra. The fluid collection device includes a porous material at least partially disposed in the chamber, the porous material having a body and a raised portion extending longitudinally along the body and through the opening.

In an embodiment, a fluid collection system is disclosed. The fluid collection system includes a fluid storage container configured to hold a fluid. The fluid collection system includes a fluid collection device fluidly coupled to the fluid storage container. The fluid collection device of the system includes a fluid impermeable barrier having an outer surface and an inner surface, the inner surface at least partially defining a chamber, the fluid impermeable barrier also defining an opening extending therethrough, the opening configured to be positioned adjacent to a female urethra; and a porous material at least partially disposed in the chamber, the porous material having a body and a raised portion extending longitudinally along the body and through the opening. The fluid collection system includes a conduit including an inlet and an outlet, the outlet being fluidly coupled to the fluid storage container and the inlet being positioned in the chamber. The fluid collection system includes a vacuum source fluidly coupled to one or more of the fluid storage container or the fluid collection device via the conduit, the vacuum source configured to draw fluid from the fluid collection device via the conduit.

In an embodiment, a method to collect fluid is disclosed. The method includes positioning a raised portion of a fluid collection device adjacent to a female urethra. The fluid collection device of the method includes a fluid impermeable barrier having an outer surface and an inner surface, the inner surface at least partially defining a chamber, the fluid impermeable barrier also defining an opening extending therethrough, the opening configured to be positioned adjacent to a female urethra; a porous material at least partially disposed in the chamber, the porous material having a body and the raised portion extending longitudinally along the body and through the opening; and a conduit including an inlet and an outlet, the outlet being fluidly coupled to a fluid storage container and the inlet being positioned in the chamber. The method includes receiving fluid from the female urethra into the fluid collection device. The method includes applying suction with a vacuum source fluidly connected to the conduit effective to suction the fluid from the chamber via the conduit.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection device, according to an embodiment.

FIG. 1B is a cross-sectional view of the fluid collection device of FIG. 1A along the plane A-A, according to at least some embodiments.

FIG. 1C is an exploded view of the fluid collection device of FIG. 1A, according to at least some embodiments.

FIG. 1D is a cross-sectional view of the fluid collection device of FIG. 1A taken along the plane B-B, according to at least some embodiments.

FIG. 2 is a cross-sectional view of the fluid collection device of FIG. 1A along the plane A-A, according to at least some embodiments.

FIG. 3 is a cross-sectional view of the fluid collection device of FIG. 1A along the plane A-A, according to at least some embodiments.

FIG. 4 is a cross-sectional view of the fluid collection device of FIG. 1A along the plane A-A, according to at least some embodiments.

FIG. 5 is a cross-sectional view of the fluid collection device of FIG. 1A along the plane A-A positioned on a wearer for use, according to at least some embodiments.

FIG. 6 is a block diagram of a system for fluid collection, according to an embodiment.

FIG. 7 is a flow diagram of a method to collect fluid, according to an embodiment.

### DETAILED DESCRIPTION

Embodiments disclosed herein are related to devices, systems, and methods of using fluid collection devices and systems. The fluid collection devices, systems, and methods herein include raised portions on the fluid collection device to aid in positioning and retention of the fluid collection device as well as to prevent leakage of urine at the interface of the fluid collection device and the tissue of the wearer.

The fluid collection devices herein include a fluid impermeable barrier that at least partially defines a chamber. The fluid impermeable barrier also defines an opening extending therethrough that is configured to be positioned adjacent to a female urethra. The fluid collection devices includes a porous material at least partially disposed within the chamber and having a raised portion that fits between the labia proximate to the urethra. The fluid collection devices may also include a conduit disposed in the chamber. The fluid collection devices disclosed herein are configured to collect fluid(s) from an individual. The fluid(s) collected by the fluid collection devices may include at least one of urine, vaginal discharge, blood, sweat, or other bodily fluids.

By providing porous material with the raised portion the fluid collection device is retained in position on the wearer with the raised portion fitting between the labia. Additionally, the raised portion of the porous material closely engages with the urethra of the wearer to prevent leakage of fluids from the user. Accordingly, the fluid collection devices disclosed herein are sized and shaped to collect fluid(s) from an individual while preventing leakage, and providing a secure fit on the wearer.

The fluid collection devices disclosed herein are configured to be used in fluid collection systems to collect and remove fluids from the wearer of the fluid collection device.

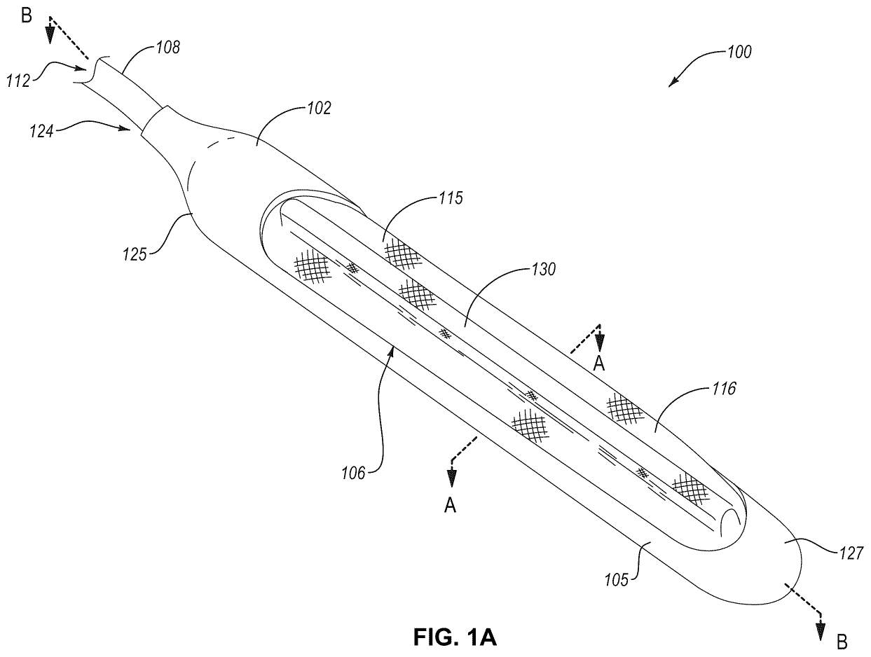

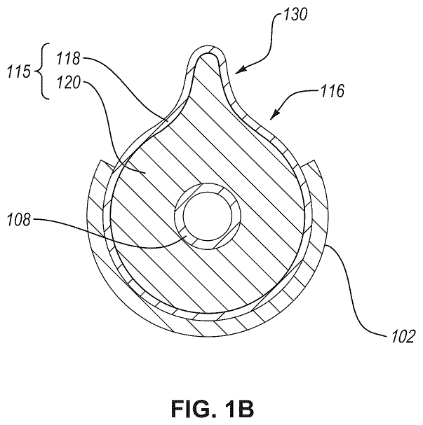

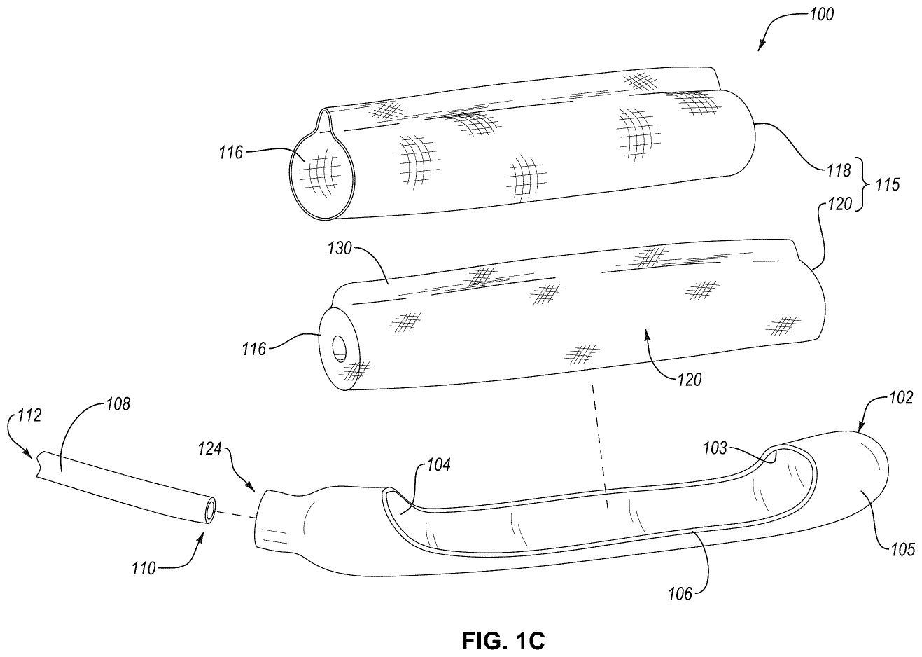

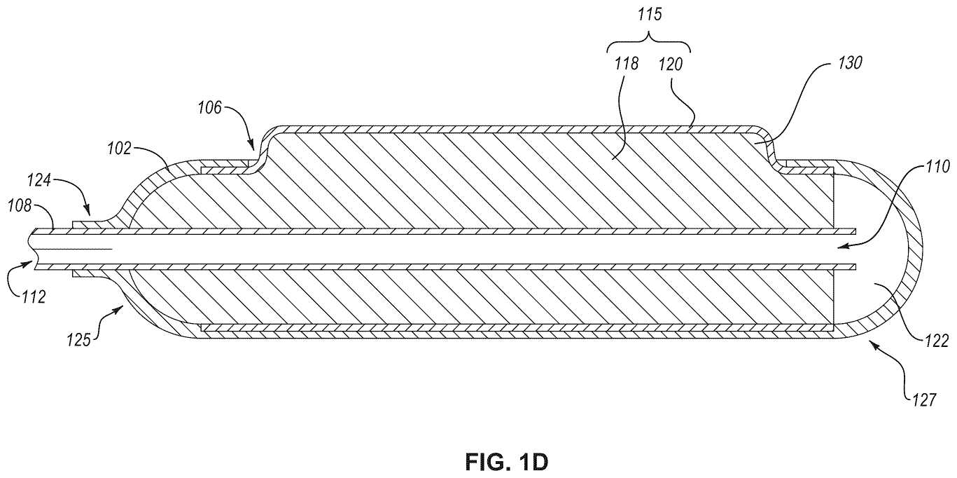

FIG. 1A is an isometric view of a fluid collection device, according to an embodiment. FIG. 1B is a cross-sectional view of the fluid collection device of FIG. 1A along the plane A-A, according to at least some embodiments. FIG. 1C is an exploded view of the fluid collection device of FIG. 1A, according to at least some embodiments. FIG. 1D is a cross-sectional view of the fluid collection device of FIG. 1A taken along the plane B-B, according to at least some embodiments. The fluid collection device 100 is an example of a female fluid collection device for receiving and collecting fluid(s) from a female. The fluid collection device 100 includes a fluid impermeable barrier 102, porous material 115 at least partially disposed in a chamber 104 within the fluid impermeable barrier 102, and an optional conduit 108 at least partially disposed within the chamber 104. The porous material 115 includes a raised portion 130 which extends from the chamber 104. The raised portion 130 aids in positioning and retaining the fluid collection device an ideal position on a wearer and prevents leakage from the device by fitting between the labia (e.g., labia majora) of the wearer to sit near the urethra of the wearer during use.

The fluid impermeable barrier 102 at least partially defines a chamber 104 (e.g., interior region) and an opening 106. For example, the interior surface(s) 103 of the fluid impermeable barrier 102 at least partially defines the chamber 104 within the fluid collection device 100. The fluid impermeable barrier 102 at least temporarily retains the fluid(s) in the chamber 104. The fluid impermeable barrier 102 may be formed of any suitable fluid impermeable material(s), such as a fluid impermeable polymer (e.g., silicone, polypropylene, polyethylene, polyethylene terephthalate, thermoplastic elastomer(s), a polycarbonate, etc.), a metal film, natural rubber, another suitable material, or combinations thereof. As such, the fluid impermeable barrier 102 substantially prevents the fluid(s) from passing through the fluid impermeable barrier 102.

In an example, the fluid impermeable barrier 102 may be air permeable and liquid impermeable. In such an example, the fluid impermeable barrier 102 may be formed of a hydrophobic material that defines a plurality of pores that are air permeable but not liquid permeable. In an example, at least one or more portions of at least an outer surface of the fluid impermeable barrier 102 may be formed from a soft and/or smooth material, thereby reducing chaffing.

In some examples, the fluid impermeable barrier 102 may be tubular (ignoring the opening), such as substantially cylindrical (as shown), oblong, prismatic, or flattened tubes. The fluid impermeable barrier 102 may be sized to fit between the legs of a female user. During use, the outer surface 105 of the fluid impermeable barrier 102 may contact the wearer.

The opening 106 provides an ingress route for fluids to enter the chamber 104. The opening 106 may be defined by the fluid impermeable barrier 102, such as by an inner edge of the fluid impermeable barrier 102. For example, the opening 106 is formed in and extends through the fluid impermeable barrier 102, from the outer surface 105 to the inner surface 103, thereby enabling fluid(s) to enter the chamber 104 from outside of the fluid collection device 100. The opening 106 may be located and shaped to be positioned adjacent to a female urethra while the device is in use. At least a portion of porous material(s) (e.g., raised portion 130) disposed in the chamber 104 may be exposed through the opening 106, to receive and move (e.g., wick) fluids inwardly into the chamber 104.

The fluid collection device 100 may be positioned proximate to the female urethra and urine may enter the chamber 104 via the opening 106. When in use, the opening 106 may be an elongated shape that extends from a first location below the urethral opening (e.g., at or near the anus or the vaginal opening) to a second location above the urethral opening (e.g., at or near the top of the vaginal opening or the pubic region).

The opening 106 may exhibit an elongated shape because the space between the legs of a female is relatively small when the legs of the female are closed, thereby only permitting the flow of the fluid(s) along a path that corresponds to the elongated shape of the opening 106 (e.g., longitudinally extending opening). The opening 106 in the fluid impermeable barrier 102 may exhibit a length that is measured along the longitudinal axis of the fluid collection device 100 that may be at least about 10% of the length of the fluid collection device 100, such as about 25% to about 50%, about 40% to about 60%, about 50% to about 75%, about 65% to about 85%, or about 75% to about 95% of the length of the fluid collection device 100.

The opening 106 in the fluid impermeable barrier 102 may exhibit a width that is measured transverse to the longitudinal axis of the fluid collection device 100 that may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid through the porous material 115 and into the conduit 108. The opening 106 may be vertically oriented (e.g., having a major axis parallel to the longitudinal axis of the device 100). In some examples (not shown), the opening 106 may be horizontally oriented (e.g., having a major axis perpendicular to the longitudinal axis of the device 100). In an example, the fluid impermeable barrier 102 may be attached to the individual, such as adhesively attached (e.g., with a hydrogel adhesive) to the individual. According to an example, a suitable adhesive is a hydrogel layer.

The fluid collection device 100 includes the porous material 115 disposed in the chamber 104. The porous material 115 may extend across at least a portion (e.g., all) of the opening 106. At least a portion of the porous material 115 may be exposed to an environment outside of the chamber 104 through the opening 106. For example, at least the raised portion 130 may extend through the opening 106 and may be exposed to an environment outside of the chamber 104. The porous material 115 may be composed to wick any fluid away from the opening 106, thereby preventing the fluid from escaping the chamber 104. The porous or permeable properties referred to herein may be wicking, capillary action, diffusion, or other similar properties or processes, and are referred to herein as “permeable” and/or “wicking.” Such “wicking” of the porous material may not include absorption of fluid into the porous material. Put another way, substantially no absorption of fluid into the porous material may take place after the material is exposed to the fluid and removed from the fluid for a time. While no absorption is desired, the term “substantially no absorption” may allow for nominal amounts of absorption and/or solubility of fluid into the porous material (e.g., absorbency), such as less than about 30 wt % of the dry weight of the porous material, about 20%, about 10 %, about 7 wt %, about 5 wt %, about 3 wt %, about 2 wt %, about 1 wt %, or about 0.5 wt % of the dry weight of the porous material. In some examples, the porous material may include at least one absorbent or adsorbent material. The porous material 115 may also allow fluid to move or wick generally towards an interior of the chamber 104, as discussed in more detail below.

The porous material 115 includes a body 116 and the raised portion 130. The body 116 forms the predominant shape and structure of the porous material 115. For example, the body 116 may be substantially cylindrical (e.g., ignoring the raised portion 130) and the raised portion 130 may extend therefrom. For example, the raised portion 130 may extend longitudinally along the body 116 (e.g., along and away from a longitudinal axis of the body 116). The body 116 may be substantially cylindrical, ellipsoid, prismatic, or the like. As discussed in more detail below, the raised portion 130 is sized and shaped to fit between the labia (e.g., labia majora of a wearer) and place an outermost portion thereof adjacent to the urethra of the wearer.

The porous material 115 may include one or more of a fluid permeable membrane 118 or a fluid permeable support 120. The fluid permeable membrane 118 may include any fluid permeable material, such as a porous material or material that may wick the fluid. For example, the fluid permeable membrane 118 may include fabric, such as a gauze (e.g., a silk, linen, or cotton gauze), another soft fabric, or another smooth fabric. The fluid permeable membrane 118 may include spun plastic fibers (e.g., nylon), such as a spun plastic mat or bed. Forming the fluid permeable membrane 118 from gauze, soft fabric, and/or smooth fabric may reduce chaffing caused by the fluid collection device 100.

The fluid collection device 100 may include the fluid permeable membrane 118 disposed in the chamber 104. The fluid permeable membrane 118 may cover at least a portion (e.g., all) of the opening 106. The fluid permeable membrane 118 may composed to receive and move (e.g., wick) any fluid inwardly away from the opening 106, thereby preventing the fluid from escaping the chamber 104.

The fluid collection device 100 may include the fluid permeable support 120 disposed in the chamber 104. The fluid permeable support 120 is configured to support the fluid permeable membrane 118 since the fluid permeable membrane 118 may be formed from a foldable, flimsy, or otherwise easily deformable material. For example, the fluid permeable support 120 may be positioned such that the fluid permeable membrane 118 is disposed between the fluid permeable support 120 and the fluid impermeable barrier 102. As such, the fluid permeable support 120 may support and maintain the position of the fluid permeable membrane 118. The fluid permeable support 120 may include any material that may wick the fluid, such as any of the fluid permeable membrane materials disclosed herein. For example, the fluid permeable membrane material(s) may be utilized in a more dense or rigid form than in the fluid permeable membrane 118 when used as the fluid permeable support 120. The fluid permeable support 120 may be formed from any fluid permeable material that is less deformable than the fluid permeable membrane 118, such as any of the materials disclosed herein for the fluid impermeable membrane 118, in a more dense or rigid form. For example, the fluid permeable support 120 may include a porous polymer (e.g., nylon, polyester, polyurethane, polyethylene, polypropylene, etc.) structure or an open cell foam or spun plastic fibers (e.g., nylon fibers). In some examples, the fluid permeable membrane 118 may include gauze and the fluid permeable support may include spun nylon fibers. In some examples, the fluid permeable support 120 may be formed from a natural material, such as cotton, wool, silk, or combinations thereof. In such examples, the material may have a coating to prevent or limit absorption of fluid into the material, such as a water repellent coating. In some examples, the fluid permeable support 120 may be formed from fabric, felt, gauze, or combinations thereof. In some examples, the fluid permeable support 120 may be omitted from the fluid collection device 100. In some examples, the fluid permeable membrane 118 may be optional. For example, the porous material 115 may include only the fluid permeable support 120.

The fluid permeable support 120 may have a greater ability to wick or flow fluids therethrough than the fluid permeable membrane 118, such as to move the fluid inwardly from the outer surface of the fluid collection device 100. In some examples, the porous ability of the fluid permeable support 120 and the fluid permeable membrane 118 may be substantially the same.

As shown, the body 116 of the porous material 115 may form the bulk structure of the porous material 115. The raised portion 130 may extend from, or be a stand-off from, the body 116. Accordingly, the raised portion 130 may fit into areas of the anatomy of a wearer that the body 116 does not.

FIG. 1B shows that the body 116 may be substantially cylindrical and the raised portion 130 may be shaped as a cusp. In examples, the body 116 may be ellipsoid, oblong, prismatic, or flattened tube-like structures. The raised portion 130 shaped as a cusp may extend away from the body 116 a distance. For example, the raised portion 130 may extend away from the body at least 3 mm, such as 3 mm to 2.5 cm, 3 mm to 1 cm, 5 mm to 1.5 cm, 1 cm to 2 cm, at least 5 mm, less than 2.5 cm, less than 1.5 cm, or less than 1 cm. A base width of the raised portion 130 may be at least 5 mm, such as 5 mm to 2 cm, 5 mm to 1.5 cm, 1 cm to 2 cm, less than 2.5 cm, or less than 1.5 cm. The dimensions of the raised portion 130 may vary, such as based on the size and shape of the body 116.

As shown in FIGS. 1B-1D, the fluid permeable support 120 may include the raised portion 130. For example, the fluid permeable support 120 may form the raised portion 130 and the fluid permeable membrane 118 may conform thereto. The raised portion 130 may be convex having a radius of curvature. The radius of curvature of the raised portion may be smaller than the radius of curvature of the body 116. As shown, the fluid permeable membrane 118 may be disposed within the chamber 104 and extend across the opening 106. The fluid permeable support 120 is also disposed within the chamber 104 beneath the fluid permeable membrane 118, the fluid permeable support 120 includes the raised portion 130 thereon. The raised portion 130 may extend out of and protrude from the opening 106. Accordingly, the raised portion 130 extends past the fluid impermeable barrier 102. The fluid permeable membrane 118 on the raised portion 130 may likewise extend past the fluid impermeable barrier 102.

The raised portion 130 may extend along the entire longitudinal length of the porous material 115. For example, the raised portion 130 of porous material 115 may extend along the entire longitudinal length of the body 116. In examples where the raised portion 130 runs along the entire length of the porous material 115 (e.g., body), the ends of the raised portion 130 may be tucked or folded under the fluid impermeable barrier 102. In such examples, the ends of the raised portion need not be removed to fit within the chamber 104.

In some examples, the raised portion 130 may extend along less than the entire longitudinal length of the porous material 115 (e.g., the body 116). For example, the raised portion 130 of porous material 115 may extend along a first half, a second half, or a central portion of the longitudinal length of the body 116. In some examples, one or more lengths of the raised portion 130 may be removed from the porous material 115. For example, the ends of the raised portion 130 may be trimmed away from the body 116 such that the raised portion 130 only extends along the portion of the porous material 115 that is positioned in the opening 106. In some examples, the raised portion may be disposed only on the first longitudinal half of the porous material 115 that extends through the opening 106 (e.g., nearest the first end region 125). In such examples, the raised portion 130 may only be present in the region of the fluid collection device expected to the disposed near the urethra of a female wearer during use. By limiting the amount of raised portion to only the first longitudinal half, the fluid collection device may be as comfortable as possible to the wearer.

The fluid permeable membrane 118 and the fluid permeable support 120 may at least substantially completely fill the portions of the chamber 104 that are not occupied by the conduit 108. In another example, the fluid permeable membrane 118 and the fluid permeable support 120 may not substantially completely fill the portions of the chamber 104 that are not occupied by the conduit 108. In such an example, the fluid collection device 100 includes the reservoir (FIG. 1D) in the chamber 104.

The fluid collection device 100 may include the conduit 108, which extends into the chamber 104. As illustrated in FIG. 1D, the conduit 108 may be at least partially disposed in the chamber 104. The conduit 108 (e.g., a tube) includes an inlet 110 at a first end region and an outlet 112 at a second end region positioned downstream from the inlet 110. The conduit 108 may extend into the chamber 104 to any point therein. For example, the conduit 108 may be inserted into the chamber at the first end region 125 of the fluid collection device 100 and extend therethrough to the second end region 127 of the fluid collection device 100. The conduit 108 may extend into the fluid impermeable barrier 102 from the first end region 125 (e.g., proximate to the outlet 112) through to the second end region 127 (e.g., opposite the first end region 125) to a point proximate to a reservoir 122 such that the inlet 110 is in fluid communication with the reservoir 122. In some examples (not shown), the conduit 108 may enter the chamber 104 in the second end region 127 and the inlet 110 of the conduit 108 may be disposed in the second end region 127 (e.g., in the reservoir 122 or flush with fluid permeable barrier 102). The fluid collected in the reservoir 122 may be removed from the chamber 104 via the conduit 108. In some examples, the inlet 110 may be disposed at the end of the fluid permeable support 120 in the second end region 127, such as flush with the end of the fluid permeable support 120. In some examples, the inlet 110 may be disposed within the fluid permeable support 120 such between first end region 125 and the second end region 127.

The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 108 may include silicon or latex. In some examples, the conduit 108 may include one or more portions that are resilient, such as to by having one or more of a diameter or wall thickness that allows the conduit to be flexible. In some examples, the conduit 108 may be frosted or opaque (e.g., black) to obscure visibility of the fluid(s) therein.

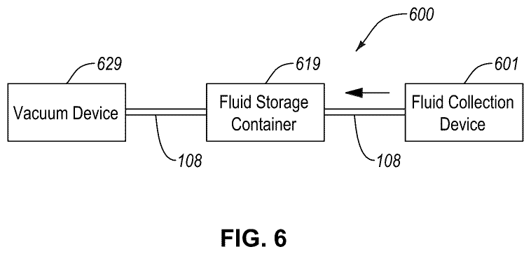

The fluid collection device 100 may be operably coupled to a vacuum source. For example, the conduit 108 fluidly couples an interior region of the chamber 104 with the fluid storage container (FIG. 6) or the vacuum source (FIG. 6). Accordingly, fluids may be removed from the chamber 104 via the conduit 108.

The fluid impermeable barrier 102, the fluid permeable membrane 118 and the fluid permeable support 120 may be configured to have the conduit 108 at least partially disposed in the chamber 104. For example, at least one of the fluid permeable membrane 118 and the fluid permeable support 120 may be configured to form a space that accommodates the conduit 108. The fluid impermeable barrier 102 may define an aperture sized to receive the conduit 108. The at least one conduit 108 may be disposed in the chamber 104 via the aperture. The apertures may be configured to form an at least substantially fluid tight seal against the conduit 108 or the at least one tube thereby substantially preventing the fluid(s) from escaping the chamber 104. The fluid collected in the fluid collection device 100 may be removed from the chamber 104 via the conduit 108.

The porous material 115 (e.g., fluid permeable membrane 118 and the fluid permeable support 120) may not substantially completely fill the portions of the chamber 104 that are not occupied by the conduit 108. The fluid collection device 100 may include the reservoir 122 therein. As shown, the reservoir 122 is a substantially unoccupied portion of the chamber 104. The reservoir 122 may be defined between the fluid impermeable barrier 102 and the porous material 115 (e.g., one or both of the fluid permeable membrane 118 and the fluid permeable support 120). The fluid(s) emitted by the wearer may be wicked into the chamber 104 by the porous material 115 (initially via the raised portion 130) and may flow through the fluid permeable membrane 118 and/or fluid permeable support 120 to the reservoir 122. The fluid impermeable barrier 102 may retain the fluid(s) in the reservoir 122. The reservoir 122 may be located in a portion of the fluid collection device expected to be positioned in a gravimetrically low point of the fluid collection device when worn by a user. While depicted in the second end region 127, the reservoir 122 may be located in any portion of the chamber 104 such as the first end region 125.

In an example, the fluid impermeable barrier 102 may be air permeable and liquid impermeable. In such an example, the fluid impermeable barrier 102 may be formed of a hydrophobic material that defines a plurality of pores that are air permeable but not liquid permeable. In an example, at least one or more portions of at least an outer surface of the fluid impermeable barrier 102 may be formed from a soft and/or smooth material, thereby reducing chaffing.

In some examples, the fluid collection device 100 may include multiple reservoirs, such as a first reservoir that is located at the portion of the chamber 104 closest to the inlet 110 (e.g., second end region 127) and a second reservoir that is located at the portion of the of the chamber 104 that is closest to the outlet 112 (e.g., first end region 125). In another example, the fluid permeable support 120 is spaced from at least a portion of the conduit 108 and the reservoir 122 may be the space between the fluid permeable support 120 and the conduit 108.

The fluid impermeable barrier 102, the fluid permeable membrane 118 and the fluid permeable support 120 may be configured to have the conduit 108 at least partially disposed in the chamber 104. For example, at least one of the fluid permeable membrane 118 and the fluid permeable support 120 may be configured to form a space that accommodates the conduit 108. In another example, the fluid impermeable barrier 102 may define an aperture 124 sized to receive the conduit 108 (e.g., at least one tube). The at least one conduit 108 may be disposed in the chamber 104 via the aperture 124. The aperture 124 may be configured to form an at least substantially fluid tight seal against the conduit 108 thereby substantially preventing the fluid(s) from escaping the chamber 104. The fluid collected in the fluid collection device 100 may be removed from the interior region of the chamber 104 via the conduit 108. As shown in FIG. 1D, the end of the conduit 108 may extend beyond the fluid permeable membrane 118 and/or fluid permeable support 120, such as into the reservoir 122. In some examples, the inlet 110 may not extend into the reservoir 122. In such examples, the inlet 110 may be disposed within the porous material 115 (fluid permeable membrane 118 and/or fluid permeable support 120) or at a terminal end thereof. For example, an end of the conduit 108 may be coextensive with or recessed within the fluid permeable membrane 118 and/or fluid permeable support 120.

Locating the inlet 110 at or near a location expected to be the gravimetrically low point of the chamber 104 when worn by a user enables the conduit 108 to receive more of the fluid(s) than if inlet 110 was located elsewhere and reduce the likelihood of pooling (e.g., pooling of the fluid(s) may cause microbe growth and foul odors). For instance, the fluid(s) in the fluid permeable membrane 118 and the fluid permeable support 120 may flow in any direction due to capillary forces. However, the fluid(s) may exhibit a preference to flow in the direction of gravity, especially when at least a portion of the fluid permeable membrane 118 and/or the fluid permeable support 120 is saturated with the fluid(s). Accordingly, one or more of the inlet 110 or the reservoir 122 may be located in the second end region 127.

Other embodiments of fluid impermeable barriers, fluid permeable membranes, fluid permeable supports, chambers, conduits and their shapes and configurations are disclosed in U.S. Pat. No. 10,973,678 filed on Jun. 2, 2017; U.S. Pat. No. 10,390,989 filed on Sep. 8, 2016; and U.S. Pat. No. 10,226,376 filed on Jun. 1, 2017, the disclosure of each of which is incorporated herein, in its entirety, by this reference.

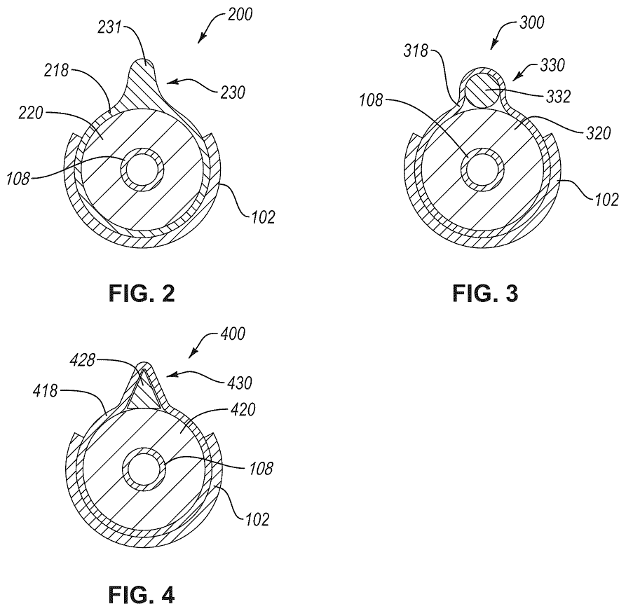

While shown as being formed in the fluid permeable support 120 and having a cusp shape, the raised portion 130 may differ in one or more of shape or material make-up than is depicted in FIGS. 1A-1D. FIG. 2 is a cross-sectional view of a fluid collection device 200, according to at least some embodiments. The fluid collection device 200 includes fluid impermeable barrier 102, fluid permeable membrane 218, fluid permeable support 220, conduit 108, and raised portion 230. The fluid collection device 200 may be similar or identical to the fluid collection device 100 in one or more aspects. For example, one or more components or portions of the fluid collection device 200 may be similar in one or more of size, shape, material composition, or relationship of components to the corresponding components or portions of the fluid collection device 100, in one or more aspects. The fluid permeable membrane 218 may be similar or identical to the fluid permeable membrane 118, in one or more aspects. The fluid permeable support 220 may be similar or identical to the fluid permeable support 120 in one or more aspects.

As shown, the raised portion 230 may be formed by the fluid permeable membrane 218. For example, the fluid permeable membrane 218 may include a shaped portion 231 (e.g., cusp shape) therein to form the raised portion 230. The raised portion 230 may be formed as a portion of stitching, quilting, or extra material in the fluid permeable membrane 218. The raised portion 230 may be similar or identical to the raised portion 130 in one or more aspects (e.g., dimension or position). In such examples, the raised portion 230 may be softer or more compliant than raised portions formed from the fluid permeable support (e.g., the raised portion 130). Such examples may be more comfortable for wearers than examples where the raised portion is formed from the fluid permeable support.

In some examples, the raised portion may not be shaped as a cusp. FIG. 3 is a cross-sectional view of a fluid collection device 300, according to at least some embodiments. The fluid collection device 300 includes fluid impermeable barrier 102, fluid permeable membrane 318, fluid permeable support 320, conduit 108, and the raised portion 330. As shown, the raised portion 330 includes a lobe. The fluid collection device 300 may be similar or identical to the fluid collection device 100 or 200 in one or more aspects. For example, one or more components or portions of the fluid collection device 300 may be similar in one or more of size, shape, material composition, or relationship of components to the corresponding components or portions of the fluid collection device 100 or 200, in one or more aspects. The fluid permeable membrane 318 may be similar or identical to the fluid permeable membrane 118, in one or more aspects. The fluid permeable support 320 may be similar or identical to the fluid permeable support 120 in one or more aspects.

As shown, the raised portion 330 may be formed as a lobe 332, such as a lobe of one or more of the fluid permeable support 320 or the fluid permeable membrane 318. For example, a main body of the fluid permeable support 320 may include a portion standing off of the body that is shaped as the lobe 332 to form at least part of the raised portion 330. The lobe 332 may be formed by pinching a portion of the fluid permeable support 320 to form a neck between the lobe and the main body of the fluid permeable support 320. The neck may be stitched to form the lobe 332. Accordingly, the lobe 332 stands off of the body of fluid permeable support 320. In some examples, the lobe 332 and the body of the fluid permeable support 320 may be integrally formed such as by molding or extrusion. The raised portion 330 may be similar or identical to the raised portion 130 or 230 in one or more aspects (e.g., dimension(s), position, material composition). The fluid permeable membrane 318 is disposed over the body of the fluid permeable support 320 and the lobe 332. Accordingly, the outer, wearer-facing surface of the device 300 has a relatively smooth outer surface without discontinuities introduced by the neck between the lobe 332 and the body of the fluid permeable support 320. In some examples, the lobe 332 may be a separate portion of fluid permeable support disposed on the main body of the fluid permeable support 320.

In some examples, the raised portion may be a separate portion of fluid permeable support bonded to the main body of the fluid permeable support. FIG. 4 is a cross-sectional view of a fluid collection device, according to at least some embodiments. The fluid collection device 400 includes fluid impermeable barrier 102, fluid permeable membrane 418, fluid permeable support 420, conduit 108, and the raised portion 430. As shown, the raised portion 430 includes a separate portion 428 of fluid permeable support material. The fluid collection device 400 may be similar or identical to the fluid collection device 100, 200, or 300 in one or more aspects. For example, one or more components or portions of the fluid collection device 400 may be similar in one or more of size, shape, material composition, or relationship of components to the corresponding components or portions of the fluid collection device 100, 200, or 300, in one or more aspects. The fluid permeable membrane 418 may be similar or identical to the fluid permeable membrane 118, in one or more aspects. The fluid permeable support 420 may be similar or identical to the fluid permeable support 120 in one or more aspects.

As shown, the raised portion 430 may be formed as the separate portion 428 of fluid permeable support material from the body of the fluid permeable support 420. The separate portion 428 may be made of the same fluid permeable support material as the body of the fluid permeable support 420 or a different fluid permeable support material (e.g., a material with lesser wicking or softer feel than the body). For example, the separate portion 428 may include a portion fluid permeable support material separate from the material of the main body. The separate portion 428 may be shaped as a cusp, lobe, or the like. The separate portion 428 may be adhered to the main body of the fluid permeable support 420 such as by gluing or heat welding. In some examples, the separate portion 428 may be disposed within the fluid permeable membrane 418 and the fluid permeable membrane 418 may be disposed over the fluid permeable support 420. In such examples, the fluid permeable membrane 418 may be disposed between the main body of the fluid permeable support 420 and the separate portion 428. The raised portion 430 may be similar or identical to the raised portion 130, 230, 330 in one or more aspects (e.g., dimension(s), position, material composition).

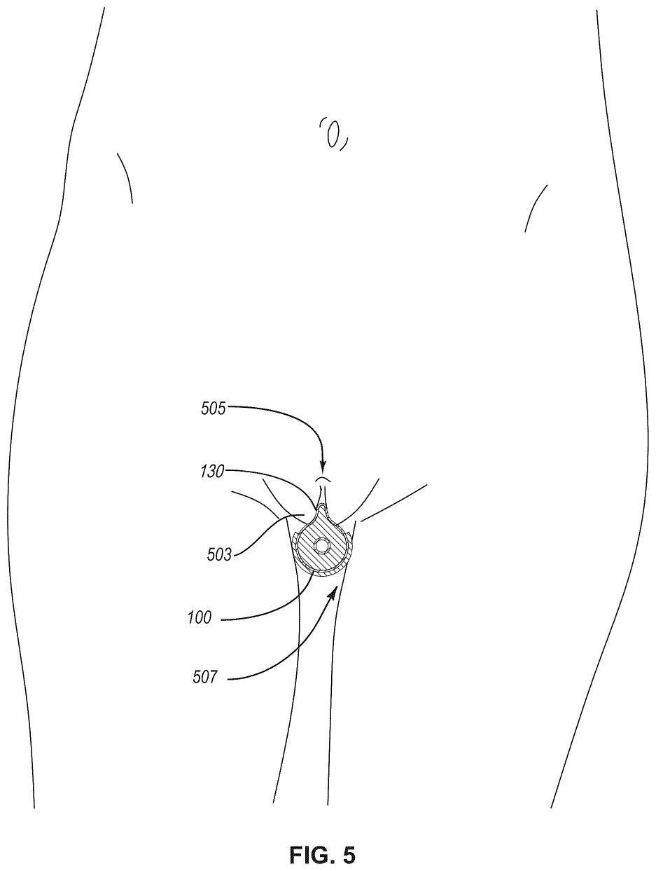

FIG. 5 is a cross-sectional view of the fluid collection device of FIG. 1A along the plane A-A positioned on a wearer for use, according to at least some embodiments. As shown, the raised portion 130 of the fluid collection device 100 fits between the labia 503 of the wearer 500. The height of the raised portion 130 is selected to place an outermost surface of the raised portion adjacent to the urethra 505 of the wearer 500. Accordingly, urine expelled by the wearer 500 immediately interfaces with the raised portion 130 and wicks inwardly toward the center of the device 100. The fluid collection devices disclosed herein limit and prevent leakage of urine due to a poor interface between the fluid collection device and the wearer.

Additionally, raised portion 130 increases the surface area of the device 100 in contact with the wearer 500, which helps maintain the device 100 in an ideal position during use. For example, the raised portion 130 friction between device 100 and the labia and mucosal membrane in the urethral region of the wearer 500. During use, the device 100 may rub against the thighs 507 of the wearer. If the wearer 500 moves, the device 100 may be subjected to rotational or translational forces in one direction or another due to contact with the thighs 507. The raised portion 130 also helps the device 100 resist rotation and translation by providing a structural interface between the labia (e.g., labia majora) and the device 100.

Any of the fluid collection devices disclosed herein may be utilized in a fluid collection system. FIG. 6 is a block diagram of a system 600 for fluid collection, according to an embodiment. The system 600 includes a fluid collection device 601, a fluid storage container 619, and a vacuum source 629. The fluid collection device 601, the fluid storage container 619, and the vacuum source 629 may be fluidly coupled to each other via one or more conduits 108. For example, fluid collection device 601 may be operably coupled to one or more of the fluid storage container 619 or the (portable) vacuum source 629 via the conduits 108.

The fluid collection device 601 may be similar or identical to any of the fluid collection devices disclosed herein. For example, the fluid collection device 601 may include a fluid impermeable barrier defining an opening and porous material including a raised portion protruding from the opening, as disclosed herein. The fluid collection device 601 may include the conduit 108 including an inlet and an outlet as disclosed herein. The outlet may be fluidly coupled to the fluid storage container 619 and the inlet may be positioned in the fluid collection device 601 such as in a portion of the chamber therein selected to be at a gravimetrically low point of the fluid collection device 601 when worn by a user (e.g., reservoir).

The conduit 108 is coupled to and at least partially extends between one or more of the fluid storage container 619 and the vacuum source 629. In an example, the conduit 108 is directly connected to the vacuum source 629. In such an example, the conduit 108 may extend from the fluid collection device 601 by at least one foot, at least three feet, at least 5 feet, or at least ten feet. In another example, the conduit 108 may be indirectly connected to at least one of the fluid storage container 619 and the vacuum source 629. In some examples, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet (110, FIGS. 1A-C) and the outlet (112, FIGS. 1A-C) may fluidly couple (e.g., directly or indirectly) the vacuum source 629 to the chamber (104 or the reservoir 122 therein, FIGS. 1A-C), such as via one or more connectors thereon. In an example, the inlet and/or the outlet may form a male connector. In another example, the inlet and/or the outlet may form a female connector. In an example, the inlet and/or the outlet may include ribs that are configured to facilitate secure couplings. In an example, the inlet and/or the outlet may form a tapered shape. In an example, the inlet and/or the outlet may include a rigid or flexible material.

Fluid (e.g., urine or other bodily fluids) collected in the fluid collection device 601 may be removed from the fluid collection device 601 via the conduit 108. For example, the first open end of the conduit 108 in the fluid collection device 601 is fluidly connected to the second open end of the conduit 108 which may be disposed in the fluid storage container 619, or the vacuum source 629. The suction force may be introduced into the interior region of the fluid collection device 601 via the first open end of the conduit 108 responsive to a suction (e.g., vacuum) force applied at the second end of the conduit 108. The suction force may be applied to the second open end of the conduit 108 by the vacuum source 629 either directly or indirectly. As the vacuum source 629 applies a vacuum/suction in the conduit 108, the fluid(s) in the chamber (e.g., at the second end region such as in the reservoir 122) of the fluid collection device 601 are drawn into the inlet and out of the fluid collection device 601 via the conduit 108.

The suction force may be applied indirectly via the fluid storage container 619. For example, the second open end (e.g., outlet) of the conduit 108 may be disposed within the fluid storage container 619 and an additional conduit 108 may extend from the fluid storage container 619 to the vacuum source 629. Accordingly, the vacuum source 629 may indirectly apply suction to the fluid collection device 601 via the fluid storage container 619. In such examples, the vacuum source 629 may provide a vacuum/suction through the fluid storage container to the fluid collection device to provide suction in the chamber of the fluid collection device. As the fluid is drained from the chamber of the fluid collection device 601, the fluid may travel through the first section of conduit 108 to the fluid storage container 619 where it may be retained.

In some examples, the suction force may be applied directly via the vacuum source 629. For example, the second open end of the conduit 108 may be disposed within the vacuum source 629. An additional conduit 108 may extend from the vacuum source 629 to a point outside of the fluid collection device 601, such as to the fluid storage container 619. In such examples, the vacuum source 629 may be disposed between the fluid collection device 601 and the fluid storage container 619. In examples, the fluid storage container 619 may include a bag (e.g., drainage bag), a bottle or cup (e.g., collection jar), a canister, or any other enclosed container for storing bodily fluids such as urine. In some examples, the fluid storage container 619 may be a rigid container.

The vacuum source 629 may include one or more of a manual vacuum pump, and electric vacuum pump, a diaphragm pump, a centrifugal pump, a displacement pump, a magnetically driven pump, a peristaltic pump, or any pump configured to produce a vacuum. In examples, the vacuum source 629 may be powered by one or more of a power cord (e.g., connected to a power socket), one or more batteries, or even manual power (e.g., a hand operated vacuum pump). The vacuum sources 629 disclosed herein may include one or more of a switch, a button, a plug, a remote, or any other device suitable to activate the vacuum source 629.

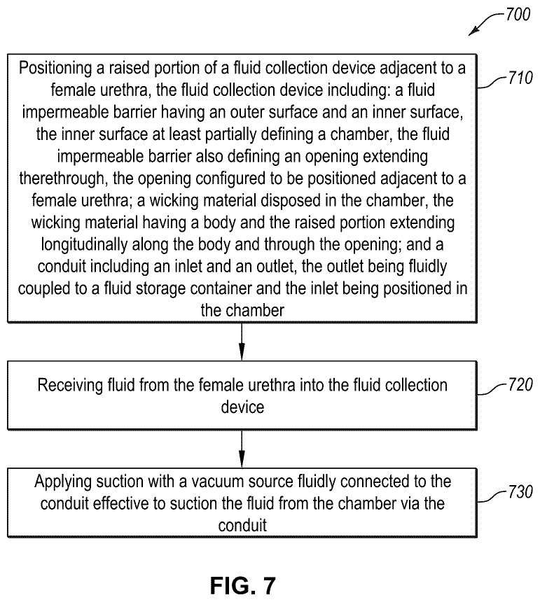

FIG. 7 is a flow diagram of a method to collect fluid, according to an embodiment. The method 700 includes act 710, which recites “positioning a raised portion of a fluid collection device adjacent to a female urethra, the fluid collection device including a fluid impermeable barrier having an outer surface and an inner surface, the inner surface at least partially defining a chamber, the fluid impermeable barrier also defining an opening extending therethrough, the opening configured to be positioned adjacent to a female urethra; a porous material disposed in the chamber, the porous material having a body and the raised portion extending longitudinally along the body and through the opening; and a conduit including an inlet and an outlet, the outlet being fluidly coupled to a fluid storage container and the inlet being positioned in the chamber.” Act 710 may be followed by act 720, which recites “receiving fluid from the female urethra into the fluid collection device.” Act 720 may be followed by act 730, which recites “applying suction with a vacuum source fluidly connected to the conduit effective to suction the fluid from the chamber via the conduit.”Acts 710, 720, 730 of the method 700 are for illustrative purposes. For example, the act 710, 720, 730 of the method 700 may be performed in different orders, split into multiple acts, modified, supplemented, or combined. In an example, one or more of the acts 710, 720, 730 of the method 700 may be omitted from the method 700. For example, the act 730 of applying suction with a vacuum source fluidly connected to the conduit effective to suction the fluid from the chamber via the conduit may be omitted. The method 700 may use any of the fluid collection devices and/or fluid collection systems disclosed herein. For example, any of the acts 710, 720, or 730 may include using any of the fluid collection devices or systems disclosed herein.

Act 710 recites “positioning a raised portion of a fluid collection device adjacent to a female urethra, the fluid collection device including a fluid impermeable barrier having an outer surface and an inner surface, the inner surface at least partially defining a chamber, the fluid impermeable barrier also defining an opening extending therethrough, the opening configured to be positioned adjacent to a female urethra; a porous material disposed in the chamber, the porous material having a body and the raised portion extending longitudinally along the body and through the opening; and a conduit including an inlet and an outlet, the outlet being fluidly coupled to a fluid storage container and the inlet being positioned in the chamber.” The fluid collection device or components thereof may be similar or identical to any of the fluid collection devices (e.g., 100-400) disclosed herein, in one or more aspects.

Positioning a raised portion of a fluid collection device adjacent to a female urethra may include positioning the opening over the female urethra, such as positioning a longitudinally extending opening of the fluid collection device over the female urethra. Positioning a raised portion of a fluid collection device adjacent to a female urethra may include positioning the raised portion between the labia (e.g., labia majora) of a wearer. Positioning a raised portion of a fluid collection device adjacent to a female urethra may include positioning a longitudinally extending raised portion longitudinally between the labia of the wearer.

In some examples, act 710 may include positioning the raised portion of the fluid collection device adjacent to a female urethra such that the raised portion abuts the female urethra. Positioning a raised portion of a fluid collection device adjacent to a female urethra may include positioning the raised portion within 2 cm of the urethra of the female wearer, such as 0.1 mm to 2 cm, 0.1 mm to 5 mm, 5 mm to 1 cm, 1 cm to 2 cm, less than 1.5 cm, less than 1 cm, less than 5 mm, more than 0.1 mm, or more than 1 mm away from the urethra of the female wearer, between the labia of the wearer. Positioning a raised portion of a fluid collection device adjacent to a female urethra may include positioning the fluid collection device between the thighs of the wearer.

The act 720 of receiving fluid from the female urethra into the fluid collection device may include receiving the fluid in the porous material. The act 720 of receiving fluid from the female urethra into the fluid collection device may include moving (e.g., wicking) the fluid(s) from the urethra via the porous material, such as via a fluid permeable membrane and a fluid permeable support. Receiving fluid from the female urethra into the fluid collection device may include wicking or otherwise moving (e.g., via gravity flow or suction) the fluid into the chamber of the fluid collection device via the porous material. The act 720 of receiving fluid from the female urethra into the fluid collection device may include moving (e.g., wicking) the fluid(s) from the raised portion into the body of the porous material (e.g., fluid permeable support). For example, the act 720 of receiving fluid from the female urethra into the fluid collection device may include wicking or otherwise moving the fluid(s) away from the opening using the raised portion, a fluid permeable membrane, and a fluid permeable support. In some examples, the act 720 may include flowing the fluid towards a portion of the chamber of the fluid collection device that is fluidly coupled to an inlet of a conduit (which may be in fluid communication a vacuum source). For instance, act 720 may include flowing the fluid(s) to a substantially unoccupied portion of the chamber (e.g., a reservoir), to a gravimetrically low point of the chamber, etc. In some examples, receiving fluid(s) from the female urethra into a chamber of the fluid collection device may include wicking or otherwise moving the fluid (e.g., urine) into the chamber via the fluid permeable membrane and fluid permeable support of the fluid collection device. For example, moving the fluid into the chamber via the fluid permeable membrane and fluid permeable support may include wicking or flowing (e.g., gravity flow or suction) urine into the reservoir in the fluid collection device.

The act 730 of applying suction with a vacuum source fluidly connected to the conduit effective to suction the fluid from the chamber via the conduit may include using any of the vacuum sources disclosed herein. The vacuum source may be spaced from the fluid collection device, such as via one or more portions of conduit. In some examples, a fluid storage container is disposed between the vacuum source and the fluid collection device. The act 730 may include activating the vacuum source (e.g., suction device) in fluid communication with the inlet of the conduit in the fluid collection device. In some examples, activating the vacuum source may include supplying power to the vacuum source by one or more of flipping an on/off switch, pressing a button, plugging the vacuum source into a power outlet, putting batteries into the vacuum source, etc. In some examples, the vacuum source may include a hand operated vacuum pump and applying suction with a vacuum source may include manually operating the hand operated vacuum pump effective to suction the fluid(s) from the chamber via the conduit.

In some examples, applying suction with a vacuum source effective to suction the fluid(s) from the chamber via a conduit disposed therein and fluidly coupled to the vacuum source may be effective to remove at least some fluid (e.g., urine) from the chamber (e.g., interior region) of the fluid collection device. In some examples, applying suction with a vacuum source effective to suction the fluid(s) from the chamber via a conduit disposed therein and fluidly coupled to the vacuum source may be effective to transfer at least some of the fluid from the chamber of the fluid collection device to a fluid storage container (e.g., a bottle or bag). In some examples, applying suction with a vacuum source effective to suction the fluid(s) from the chamber may include removing fluid from one or more of a reservoir, fluid permeable support, or fluid permeable membrane of the fluid collection device.

In an example, the method 700 may include collecting the fluid(s) that are removed from the fluid collection device, such as into the fluid storage container that is spaced from the fluid collection device and fluidly coupled to the conduit. The fluid storage container may include any of the fluid storage containers disclosed herein.

The fluid collection devices, systems, and methods disclosed herein provide for secure positioning of fluid collection devices between the labia of a wearer in very close proximity to the urethra of the female wearer, which prevents leakage and dislodgement of the fluid collection device.

While various aspects and embodiments have been disclosed herein, other aspects and embodiments are contemplated. The various aspects and embodiment disclosed herein are for purposes of illustration and are not intended to be limiting.

1. (canceled)

2. A fluid collection device, comprising:
a fluid impermeable barrier having an outer surface and an inner surface, the inner surface at least partially defining a chamber, the fluid impermeable barrier also defining an opening extending therethrough, the opening configured to be positioned adjacent to a female urethra; and
a porous material at least partially disposed in the chamber, the porous material having a body and a raised portion extending from the body and through the opening, the raised portion extending along a portion of a longitudinal length of the body.

3. The fluid collection device of claim 2, wherein the raised portion includes a cusp extending away from the body.

4. The fluid collection device of claim 2, wherein the raised portion includes a lobe standing off of the body.

5. The fluid collection device of claim 2, wherein:
the body is substantially cylindrical; and
the raised portion extends away from the body at least 5 mm.

6. The fluid collection device of claim 2, wherein the porous material includes:
a fluid permeable outer layer disposed within the chamber and extending across the opening; and
a fluid permeable inner layer disposed within the chamber beneath the fluid permeable outer layer, the fluid permeable inner layer including the raised portion thereon.

7. The fluid collection device of claim 2, wherein the porous material includes:
a fluid permeable outer layer disposed within the chamber and extending across the opening;
a fluid permeable inner layer disposed within the chamber beneath the fluid permeable outer layer, the fluid permeable inner layer including the raised portion thereon; and
an intermediate layer disposed between a portion of the fluid permeable outer layer extending across the opening and an adjacent portion of the fluid permeable inner layer, the intermediate layer forming the raised portion.

8. The fluid collection device of claim 7, wherein the intermediate layer exhibits a generally circular cross-sectional shape.

9. The fluid collection device of claim 7, wherein the intermediate layer exhibits a generally triangular cross-sectional shape.

10. The fluid collection device of claim 2, wherein the raised portion only extends from a portion of the body that extends across the opening.

11. The fluid collection device of claim 10, wherein the raised portion only extends along a proximal longitudinal half of the portion of the body that extends across the opening, the proximal longitudinal half is positioned closer to an aperture defined by the fluid impermeable barrier.

12. The fluid collection device of claim 2, wherein the raised portion extends along a central portion of the body.

13. The fluid collection device of claim 2, wherein the body is substantially cylindrical and the raised portion is configured to fit into areas of an anatomy of a wearer that the body does not.

14. The fluid collection device of claim 2, further comprising the conduit; and
wherein the fluid impermeable barrier defines an aperture and the conduit extends through the aperture into the chamber.

15. The fluid collection device of claim 2, wherein the fluid impermeable barrier partially defines a reservoir.

16. A fluid collection system, comprising:
a fluid storage container configured to hold a fluid;
the fluid collection device of claim 2, the fluid collection device fluidly coupled to the fluid storage container;
the conduit including an inlet and an outlet, the outlet being fluidly coupled to the fluid storage container, the inlet positioned in the chamber; and
a vacuum source fluidly coupled to one or more of the fluid storage container or the fluid collection device via the conduit, the vacuum source configured to draw fluid from the fluid collection device via the conduit.

17. A method to collect fluid, the method comprising:
positioning a raised portion of a fluid collection device adjacent to a female urethra, the fluid collection device including:
a fluid impermeable barrier having an outer surface and an inner surface, the inner surface at least partially defining a chamber, the fluid impermeable barrier also defining an opening extending therethrough, the opening configured to be positioned adjacent to a female urethra;
a porous material at least partially disposed in the chamber, the porous material having a body and the raised portion extending from the body and through the opening, the raised portion extending along a portion of a longitudinal length of the body; and
the conduit including an inlet and an outlet, the outlet being fluidly coupled to a fluid storage container, the conduit positioned in the aperture and the inlet being positioned in the chamber; and
receiving fluid from the female urethra into the fluid collection device.

18. A fluid collection device, comprising:
a fluid impermeable barrier having an outer surface and an inner surface, the inner surface at least partially defining a chamber, the fluid impermeable barrier also defining an opening extending therethrough, the opening configured to be positioned adjacent to a female urethra, the fluid impermeable barrier defining an aperture sized to receive a conduit;
a porous material at least partially disposed in the chamber, the porous material having a body and a raised portion extending from the body and through the opening, the raised portion only extending from a portion of the body that extends across the opening;
a conduit including an inlet and an outlet, the conduit positioned in the aperture and the inlet positioned in the chamber.

19. The fluid collection device of claim 18, wherein the porous material includes:
a fluid permeable outer layer disposed within the chamber and extending across the opening; and
a fluid permeable inner layer disposed within the chamber beneath the fluid permeable outer layer, the fluid permeable inner layer including the raised portion thereon.

20. The fluid collection device of claim 18, wherein the porous material includes:
a fluid permeable outer layer disposed within the chamber and extending across the opening;
a fluid permeable inner layer disposed within the chamber beneath the fluid permeable outer layer, the fluid permeable inner layer including the raised portion thereon; and
an intermediate layer disposed between a portion of the fluid permeable outer layer extending across the opening and an adjacent portion of the fluid permeable inner layer, the intermediate layer forming the raised portion.

21. The fluid collection device of claim 18, wherein the raised portion only extends along a proximal longitudinal half of the portion of the body that extends across the opening, the proximal longitudinal half is positioned closer to an aperture defined by the fluid impermeable barrier.
